(19) United States
(12) Patent Application Publication
(10) Pub. No.: US 2003/0191137 A1
(43) Pub. Date: Oct. 9, 2003

(54) Prophylactic and therapeutic use of oltipraz as an antifibrotic and anticirrhotic agent in the liver and pharmaceutical composition containing oltipraz

(76) Inventors: Sang-Geon Kim, Seoul (KR); Keon-Wok Kang, Seoul (KR)

Correspondence Address: CANTOR COLBURN, LLP, 55 GRIFFIN ROAD SOUTH, BLOOMFIELD, CT, 06002

(21) Appl. No.: 10/240,491

(22) PCT Filed: Mar. 2, 2001
(86) PCT No.: PCT/KR01/00319

(30) Foreign Application Priority Data
Apr. 7, 2000 (KR) 2000-18134

(51) Int. Cl. A61K 31/4965; A61K 31/497

(52) U.S. Cl. 514/255.05

(57) ABSTRACT

The present invention provides a prophylactic and therapeutic use of 5-(2-pyrazinyl)-4-methyl-1,2-dithiol-3-thione (oltipraz) as an antifibrotic and anticirrhotic agent in the liver and a pharmaceutical composition containing oltipraz for treating and preventing hepatic fibrosis and cirrhosis.

Oltipraz of the invention can be used as a medicine optionally with other drugs for treating and preventing hepatic fibrosis and cirrhosis and shows an inhibiting effect of hepatic fibrosis at a relatively low dosage. Formulations using an optimal dose of oltipraz, which is provided by the invention, have a surprisingly good effect on the treatment and prevention of hepatic fibrosis and cirrhosis and are safe drugs that have a low toxicity on the human body.

### BACKGROUND OF THE INVENTION

1. Field of the Invention

The present invention relates to a prophylactic and therapeutic use of 5-(2-pyrazinyl)-4-methyl-1,2-dithiol-3-thione (oltipraz) as an antifibrotic and anticirrhotic agent in the liver and to a pharmaceutical composition comprising oltipraz as an active ingredient.

2. Description of the Related Art

The liver plays a key role in the metabolism of xenobiotics and in the metabolism of endogenous substances and is an important organ with consistent enzymatic reactions and energy metabolism. Among the many chronic diseases in Korea, hepatitis, cirrhosis, and liver cancer are the most widespread and life threatening next to cardiovascular diseases. As Korea has a relatively large population of drinkers of alcoholic beverages when compared to developed countries, and as liver damage due to binge drinking is fairly high, a lot of attention has been given to the treatment of liver diseases. Often chronic liver damage resulting from viral infection or alcohol consumption causes cirrhosis or liver cancer. In consideration of the physiological characteristics and importance of the liver, and in view of the importance of treating and preventing liver disease, demand is high for the ultimate development of therapeutic and preventive drugs against liver damage.

Various substances, including several synthetic compounds and galenical preparations, show hepatoprotective functions both in vitro and in vivo. Although it has been known that silymarin and betaine have liver protective effects as a result of the action mechanism of cytokine inhibition and an increase in the level of glutathione, a curative effect would be hard to expect because of its low effectiveness. Because no appropriate curative agents against liver disease are currently available, said agents are frequently used for clinical trials. Malotilate and its derivatives, the indication of which is the treatment of liver fibrosis, protect the liver from toxic chemicals and the possible action mechanism includes the induction of phase II conjugating enzymes and the inhibition of cytochrome P450s. However, the compounds non-selectively inhibit several cytochrome P450s and show only preventive effects.

It is known that several substituents of sulfur containing dithiolthione, which naturally occurs in cruciferous vegetables, have liver protecting effects. Among them, oltipraz was used as a curative agent against schistosomiasis with the following formula.

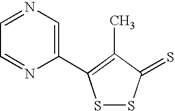

Oltipraz increases cellular thiol content and induces the expression of enzymes responsible for maintaining the glutathione (GSH) pool and detoxifying the tissue from electrophilic molecules. The activities of the following enzymes are increased by oltipraz: NAD(P)H quinone reductase, microsomal epoxide hydrolase, glutathione S-transferase (GST) and UDP glucuronyl transferase (UDP-GT). In particular, GST protects the liver from some toxic chemicals such as carbon tetrachloride or acetaminophen (Ansher S S, Dolan P, and Bueding E. Chemoprotective effects of two dithiolthiones and of butylhydroxyanisole against carbon tetrachloride and acetaminophen toxicity. 1983, Hepatology 3, 932-935).

Furthermore, oltipraz inhibits chemical carcinogenesis caused by benzo[a]pyrene, NDEA, and uracil mustard as well as aflatoxin B1-induced hepatic tumorigenesis and azoxymethane-induced colon carcinogenesis (Bolton M G, Munoz A, Jacobson L P, Groopman J D, Maxuitenko Y Y, Roebuck B D, and Kensler T W. Transient intervention with oltipraz protects against aflatoxin-induced hepatic tumorigenesis. 1993, Cancer Res. 53, 3499-3504).

The known inhibitory mechanisms of carcinogenesis by oltipraz are the following. First, oltipraz increases the level of an antioxidant, reduced GSH, in tissues. Second, it inhibits bioactivation of carcinogens by inhibiting phase I enzymes such as cytochrome P450. Third, it promotes detoxification of carcinogens by inducing phase II detoxifying enzymes including GST and UDP-GT. Fourth, oltipraz inhibits replication of the human immunodeficiency virus (HIV) type I in vitro. Fifth, it removes reactive intermediates in cells by increasing thiol levels and promotes DNA repair. It has been reported that oltipraz increases GSH levels in most tissues and removes free radicals generated by radiation or xenobiotics. It also has been known that oltipraz functions as a protective agent against radiation by helping to maintain cellular homeostasis.

With regard to the above description, more detailed information will be set forth below. Cancer is uncontrolled cell growth and differentiation presumably caused by DNA damage in the somatic cells (Cancer Biology, 3rd ed. Raymond W. Ruddon, pp. 61-95, 497-507, Oxford Press). Anticancer effects of chemical agents primarily rely on their anti-mutagenesis effects or their ability to suppress transformation into cancer cells or proliferation of cancer cells. Oltipraz has been studied as a cancer chemopreventive agent (Ansher et al., 1983; Bolton et al., 1993). The cancer chemopreventive effects of oltipraz are associated not only with the inhibition of cytochrome P450 3A, but also with the induction of phase II detoxifying enzymes. The expression of GST is increased by oltipraz in cells and animals (Clapper et al., 1994; Davidson et al., 1990), which is associated with suppression in toxicant-induced tissue injuries and carcinogenesis (Kensler et al., 1987; Maxuitenko et al., 1998). Oltipraz protects the liver against tissue damage caused by radiation (Kim et al., 1997), and GST induction, known from the prior study, means cellular adaptive response. Oltipraz also protects the liver against toxicants (Ansher et al., 1983). The inhibition of aflatoxin B1-induced carcinogenesis by oltipraz is mediated through the intervention of cytochrome P450 3A-catalyzed metabolic activation of carcinogen. According to recent clinical trials, oltipraz was effective in lowering plasma aflatoxin B1 levels in people who are high risk for contracting liver cancer. Aflatoxin B1-induced carcinogenesis in animals was also reduced by the application of oltipraz.

It has been reported that oltipraz inhibits hepatitis B virus (HBV) replication in 2.2.15 cells, which were infected with HBV DNA-containing plasmid. Therefore, oltipraz inhibits transcription of the hepatitis B virus gene, elevates p53 protein expression (Chi et al., 1998), and inhibits HIV replication (Prochaska et al., 1995).

Clinical trials on the chemopreventive effect of oltipraz against liver carcinogenesis have been conducted in China. The results show that oltipraz is weak in protecting against liver carcinogenesis. It is also known that oltipraz protects the liver against toxicant-induced hepatotoxicity, at least moderately. In addition, the safety of oltipraz has been proven in toxicity studies performed in rats and dogs (Fund. Appl. Toxicol. 1997 Jan; 35(1):9-21).

Liver fibrosis means a prepathological state in which damaged liver tissue in chronic liver diseases such as hepatitis is not repaired into normal tissue, but is converted into fibrous tissue such as collagen as part of an in vivo adaptive response. Although liver fibrosis is the outcome of an in vivo repair process in response to tissue damage, damaged liver tissue is replaced by fibrous tissue that can no longer function normally (e.g. in vivo metabolism or bile juice production). As continuous and recurring liver fibrogenesis leads to cirrhosis and eventually causes death, it is crucial to develop new drugs to treat liver fibrosis. However, as the precise mechanism of liver fibrogenesis is not known, appropriate curative drugs have not yet been developed.

Recent studies revealed that transforming growth factor-beta (TGF-β), a cytokine secreted from Kupffer and Ito cells in the liver, was an important mediator in liver fibrosis. In addition, it was reported that blocking TGF-β activity by employing TGF-β antibodies, antisense RNA, and modifications to TGF-β receptors significantly decreases liver fibrosis. However, the effects of said research have only been confirmed at the experimental level. Clinically viable drugs for liver fibrosis and cirrhosis have not been reported.

### SUMMARY OF THE INVENTION

The object of the present invention is to provide a pharmaceutical composition that maximizes the treatment effectiveness of hepatic fibrosis and cirrhosis, and that can be used as a preventive agent as well.

More specifically, the object of the present invention is to provide a use of 5-(2-pyrazinyl)-4-methyl-1,2-dithiol-3-thione (oltipraz) for the treatment and prevention of hepatic fibrosis and cirrhosis. Another object of the present invention is to provide a method of treating or preventing hepatic fibrosis and cirrhosis, which comprises administering a pharmaceutical composition comprising oltipraz as an active ingredient to a mammal.

The inventors have carried out an investigation to develop an effective drug for the treatment and prevention of hepatic fibrosis and cirrhosis and have thus found that 5-(2-pyrazinyl)-4-methyl-1,2-dithiol-3-thione (oltipraz) has a surprisingly excellent effect on the treatment and prevention of hepatic fibrosis and cirrhosis.

Thus, the present invention provides a pharmaceutical composition for treating and preventing hepatic fibrosis and cirrhosis comprising 5-(2-pyrazinyl)-4-methyl-1,2-dithiol-3-thione and a pharmaceutically acceptable excipient.

Oltipraz of the present invention can be used as a medicine for the treatment and prevention of hepatic fibrosis and cirrhosis, and it shows an inhibiting effect of hepatic fibrosis at a relatively low dosage. Formulations using an optimal dose of oltipraz, which are provided by the invention, have a surprisingly good effect on the treatment and prevention of hepatic fibrosis and cirrhosis and are safe drugs that have a low level of toxicity to the human body.

### BRIEF DESCRIPTIONS OF THE DRAWINGS

FIG. 1a is a photograph of liver tissue of a normal animal (H&E staining).

FIG. 1b is a photograph of liver tissue from the group to which oltipraz was administered (H&E staining).

FIG. 1c is a photograph of liver tissue from the group to which DMN was administered (H&E staining).

FIG. 1d is a photograph of liver tissue from the group to which DMN and oltipraz were co-administered (H&E staining).

FIG. 2a is a photograph of liver tissue from the group to which DMN was administered (Van Gieson staining).

FIG. 2b is a photograph of liver tissue from the group to which DMN and oltipraz were co-administered (Van Geison staining).

FIG. 2c is a photograph of liver tissue from the group to which DMN was administered (Masson's Trichrome staining).

FIG. 2d is a photograph of liver tissue from the group to which DMN and oltipraz were co-administered (Masson's Trichrome staining).

FIG. 3 is a photograph showing the inhibition effect of oltipraz on TGF-β 1 mRNA expression in liver tissue when DMN is administered to a rat FIG. 4 is a photograph showing the inhibition effect of oltipraz on TNF-alpha production increased by LPS in rats.

### DETAILED DESCRIPTION OF THE PRESENT INVENTION

The present inventors have made an unprecedented discovery in which oltipraz has been found to have an unexpectedly surprising effect of treating and preventing hepatic fibrosis and cirrhosis by inhibiting TGF-β production.

As shown above, it is known that oltipraz protects against hepatotoxicity and inhibits the carcinogenic process. However, no other reference had ever disclosed or reported the effect of oltipraz in the treatment and prevention of hepatic fibrosis and cirrhosis as is taught by the current invention.

Fibrosis, a preliminary stage of cirrhosis, occurs when severe damage is done to the liver and is due to by a variety of factors. Cirrhosis is partially related to carcinogenesis and notably increases the risk of liver cancer in its victims. However, the pathological mechanism of cirrhosis is clearly distinguishable from liver cancer. That is, hepatic fibrosis occurs when there is chronic and severe damage to hepatic tissue. The causative factors for liver damage include viruses, parasites, alcohol consumption, chemicals, and medicines. Hepatic fibrosis occurs through the overproduction of the extracellular matrix (e.g., type I, III and IV collagen) caused by the activation of non-parenchymal cells in hepatic tissue, such as Kupffer cells, stellate cells, etc. More specifically, fibrosis occurs due to the activation and subsequent transformation of stellate cells into myofibroblasts. The activated stellate cells then produce excess extracellular matrices. Furthermore, fibrosis and cirrhosis are clearly distinguishable as pathological phenomena apart from viral hepatitis and liver cancer. Thus, their respective treatments and preventions are also distinguishable. However, currently, there is no clinically viable drug for hepatic cirrhosis.

The present invention is based on the discovery that oltipraz, known to be effective in the prevention of liver cancer, is also effective against liver fibrosis and cirrhosis, which are completely different in their pathological mechanisms from liver cancer. These facts are proven in the experiments described below.

Oltipraz decreases the fibrosis score and Knodell score, indicators of dimethylnitrosamine (DMN) accelerated fibrosis. This coincides with exemplary tissue microscopy examinations. Additionally, upon administration, oltipraz significantly inhibits hepatotoxicity indicators such as alanine aminotransferase (ALT), aspartate aminotransferase (AST), bilirubin, and gamma-glutamyl transpeptidase (gamma-GT). This shows that oltipraz may ameliorate fibrosis by retarding their respective processes. The fibrosis inhibition mechanism of oltipraz revolves around the inhibition of TGF-β expression. According to quantitative RT-PCR results, oltipraz completely inhibits TGF-β mRNA expression accelerated by dimethylnitrosamine. This serves as evidence that oltipraz is a drug that is capable of inhibiting the genesis and progression of hepatic fibrosis and cirrhosis. Oltipraz especially shows the potential to be a superior anti-fibrotic drug because it exhibits strong anti-fibrotic effects, induces the hepatic detoxification enzyme GST, increases GSH, and exhibits radical conjugating activity. Even a low dosage of oltipraz is expected to have a satisfactory pharmacological effect.

In the present invention, the curative effects of oltipraz on hepatic fibrosis were observed in rats that had been administered with DMN in various dosages. In the results, the DMN administered group showed a four-fold increase in plasma ALT and AST activity. In comparison, when oltipraz was administered, increases in plasma ALT and AST activity were inhibited in a dosage-dependent manner. Plasma gamma-GT activity and bilirubin content are used as indicators of hepatic functionality. Oltipraz inhibited DMN-accelerated gamma-GT activity in rats by 70%-80%. On the other hand, upon DMN administration, bilirubin content increased eight-fold. After co-administering DMN and oltipraz, blood plasma bilirubin was suppressed by 65%. Also, in evaluating the fibrosis score and the Knodell score from the tissue microscopy, it was found that oltipraz blocked DMN-induced hepatic fibrosis progression remarkably. These pharmacological functions are thought to occur primarily due to the inhibition of TGF-β production by means of oltipraz, and it may be presumed that induction of antioxidant enzymes and increase in GSH contribute in part to the anti-fibrosis process by means of oltipraz.

Oltipraz may be used as a clinically viable drug that is effective in the treatment and prevention of hepatic fibrosis and cirrhosis. When the pharmaceutical composition of the present invention is put to actual use, the unit dosage forms suitable for oral administration are to be formulated and administered according to the conventions of the proper pharmaceutical field. To this end, the oral formulation comprises a hard or soft capsule, tablet, powder, etc. The oral formulation, in addition to oltipraz as the pharmacologically active agent, may contain one or more pharmacologically non-active conventional carriers. For example, the oral formulation may contain excipients such as starch, lactose, carboxymethylcellulose and kaolin; binders such as water, gelatin, alcohol, glucose, arabic gum and tragacanth gum; disintegrants such as starch, dextrine and sodium alginate; and lubricants such as stearic acid, magnesium stearate and liquid paraffin.

The daily dosage of the present invention depends on various factors such as the patient's degree of liver damage, time of onset of hepatitis, age, health, other complications, etc. However, for the average adult, oltipraz is administered once or twice a day for a total daily dosage of 10 to 1000 mg, more preferably 50 to 300 mg. However, in cases where the patient has severe liver damage or when used as an anti-recurring agent after hepatic carcinectomy, the present invention can go beyond the scope of the above pharmaceutical composition and employ even larger dosages.

The present invention seeks to use oltipraz, a superior hepatic fibrosis and cirrhosis progress inhibitor, to produce a drug with low toxicity and nearly no side effects for not only treatment purposes but also for prevention through safe, long-term use. Thus, the pharmaceutical composition of the present invention may be safely used over the long-term for the treatment and prevention of hepatic fibrosis and cirrhosis.

The present invention is explained in greater detail in the working examples below. However, the present invention is not limited to these working examples.

The present invention is explained in detail by the test examples below.

### TEST EXAMPLES

### Test Example 1

### Effect of Oltipraz on Fibrosis-1

Rats that were continually administered dimethylnitrosamine (DMN) over a period of 4 weeks displayed a four-fold increase in plasma ALT and AST activities. Upon pretreatment of 50 mg/kg oltipraz, increases in plasma ALT and AST activities were inhibited by 50% (Table 1).

Plasma gamma-GT activity and bilirubin content are used as indicators of hepatic functionality. Oltipraz inhibited increases in gamma-GT activity by 70%-80% in DMN administered rats. On the other hand, when DMN was administered, bilirubin content increased eight-fold compared to the control group. When 50 mg/kg oltipraz and DMN were simultaneously administered, the plasma bilirubin increase was inhibited by 65%.

TABLE 1
ALT, AST, gamma-GT, Bilirubin Values

| Group | ALT | AST | gamma-GT | Bilirubin |
|---|---|---|---|---|
| Control | 49 ± 2 | 113 ± 6 | 0.2 ± 0.1 | 0.2 ± 0.01 |
| DMN | 190 ± 12* | 412 ± 39* | 12.1 ± 4.1* | 0.9 ± 0.2* |
| DMN + | 116 ± 4^{#} | 246 ± 32^{#} | 2.6 ± 0.5^{#} | 0.3 ± 0.03^{#} |
| Oltipraz 50 | | | | |
| mg/kg | | | | |

Each value is represented by the average 1 standard deviation. The number of animals used ranged from 8 to 16. The significance of each group was indicated by the Newman-Keuls test of multiple analysis. The markers of significance are: *=p<0.05 compared to control, #=p<0.05 compared to DMN treated group.

### Test Example 2

### Effect of Oltipraz on Fibrosis-2

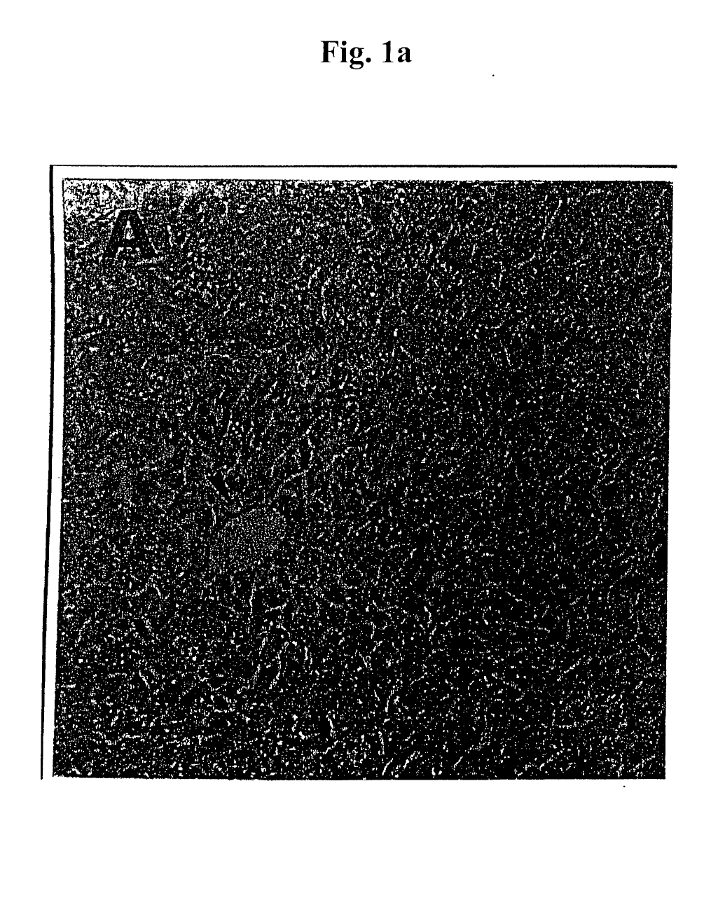

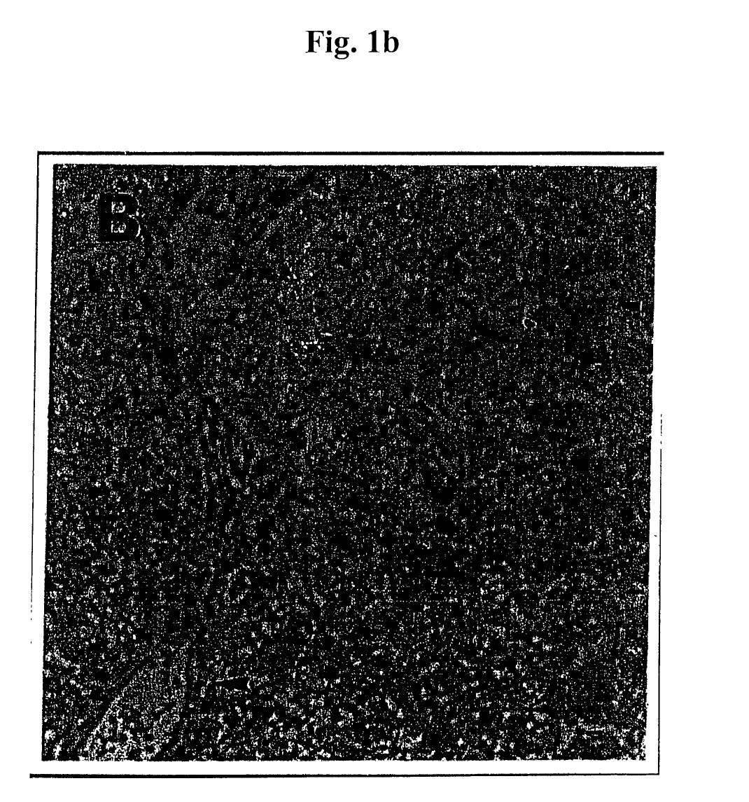

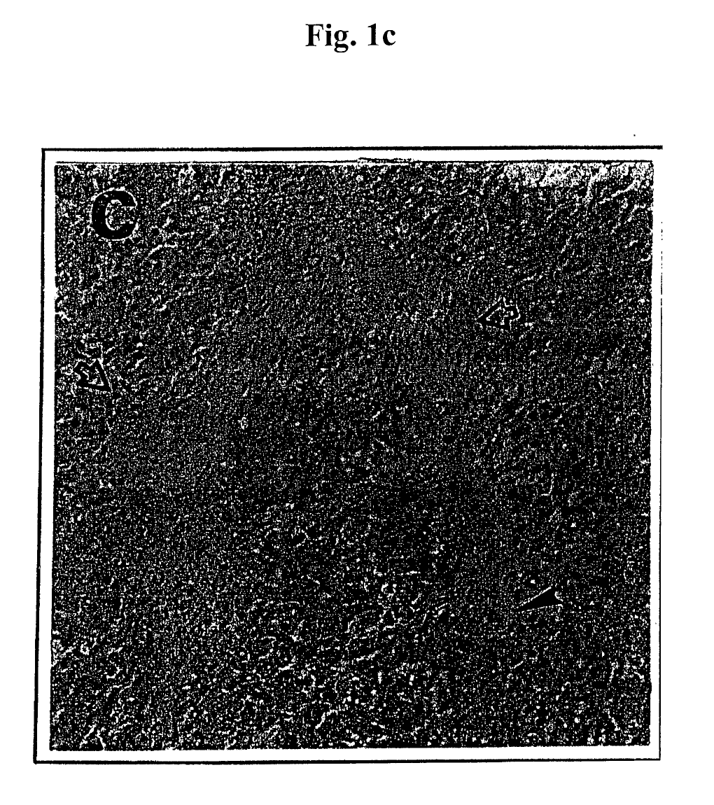

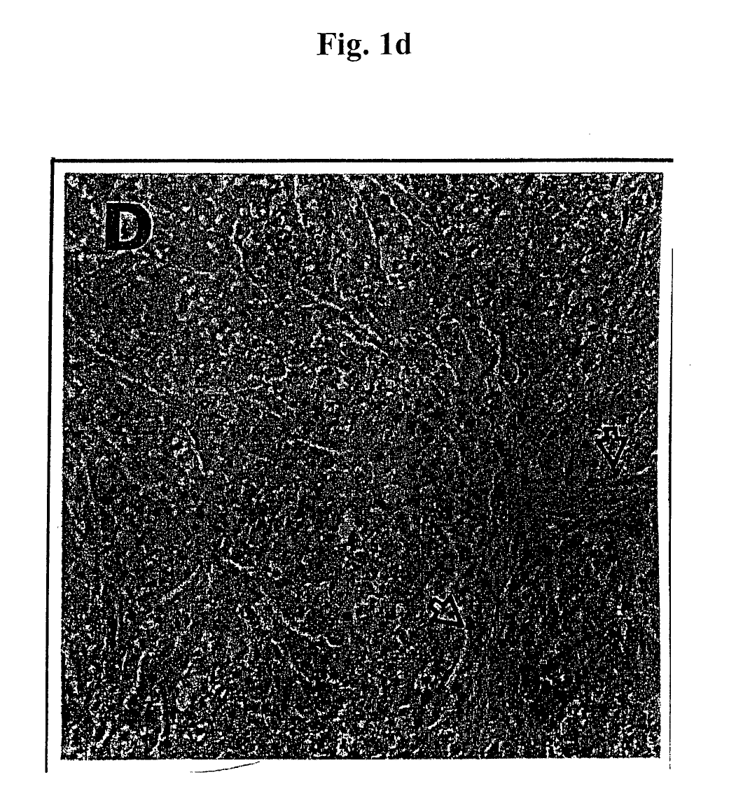

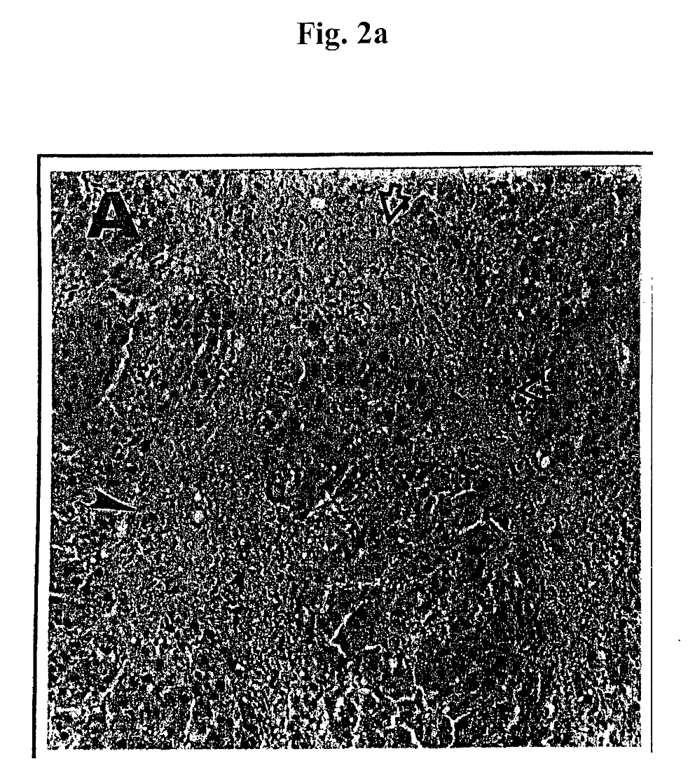

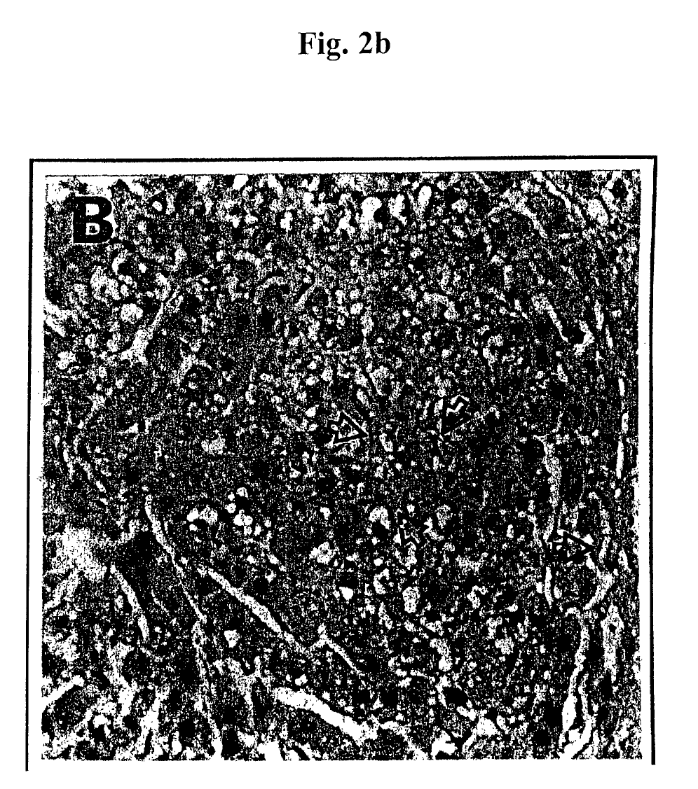

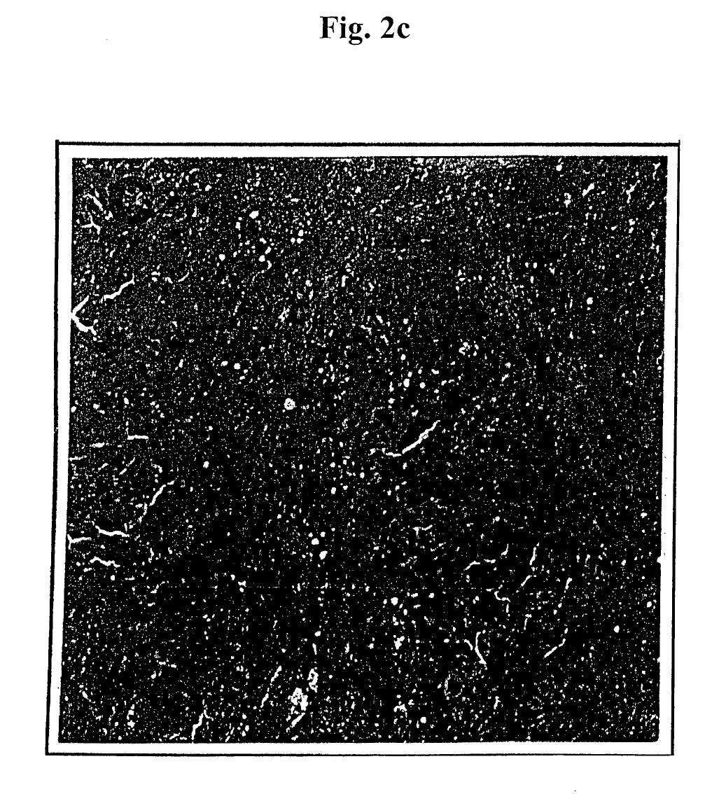

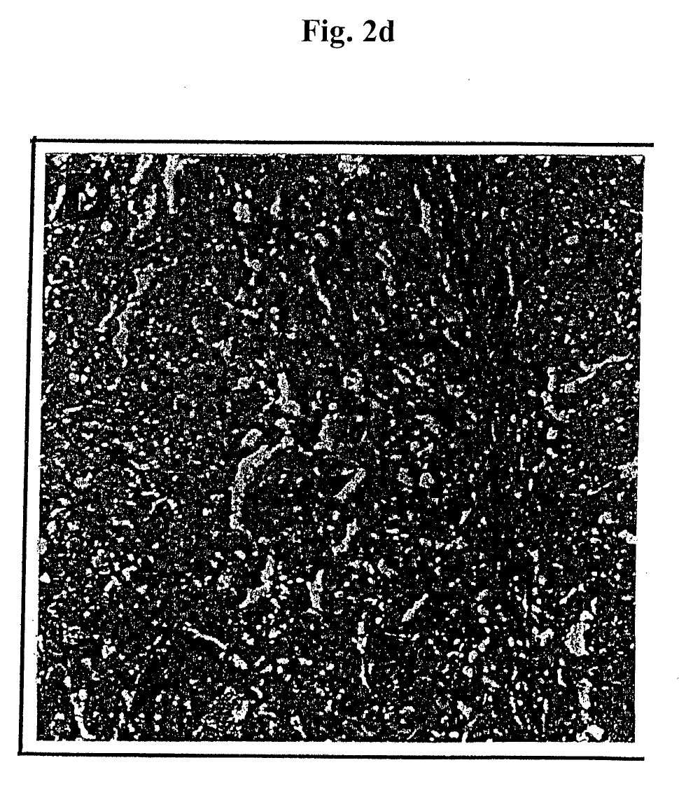

The histopathological effect of oltipraz on DMN induced hepatic fibrosis was observed in an animal test model. Clear fibrosis was observed in rats that had been administered DMN 3 times per week over a 4-week period. When oltipraz (orally administered in doses of 5-50 mg/kg doses 3 times a week over a 4-week period) and DMN were administered simultaneously, fibrosis in the hepatic tissue was reduced when compared to DMN administration alone. Hepatic tissue necrosis and fibrosis were pathologically determined through the use of hepatic tissue pathology indicators, namely, Van Gieson's staining and Masson's trichrome staining (FIGS. 1 and 2).

FIG. 1a is a photograph of liver tissue of a normal animal (H&E staining), FIG. 1b is a photograph of liver tissue from the group that was administered oltipraz (H&E staining), FIG. 1c is a photograph of liver tissue from the group that was administered DMN (H&E staining), and FIG. 1d is a photograph of liver tissue from the group that was administered both DMN and oltipraz (H&E staining). FIG. 2a is a photograph of liver tissue from the group that was administered DMN (Van Gieson staining), FIG. 2b is a photograph of liver tissue from the group that was administered both DMN and oltipraz (Van Gieson staining), FIG. 2c is a photograph of liver tissue from the group that was administered DMN (Masson's Trichrome staining), and FIG. 2d is a photograph of liver tissue from the group that was administered both DMN and oltipraz (Masson's Trichrome staining).

50 mg/kg oltipraz dose effectively ameliorated DMN induced fibrosis (Table 2). The degree of fibrosis was determined by evaluating the fibrosis and Knodell scores, which show degrees of liver damage and fibrosis. Compared to the DMN-only group, the DMN+oltipraz group showed lower fibrosis and Knodell scores, showing remedy of liver damage and fibrosis.

TABLE 2
Inhibition Effect of Oltipraz on Hepatic Tissue Fibrosis

| Group | Fibrosis Values | Knodell Values |
|---|---|---|
| Control | 0 | 0 |
| DMN | 3.7 ± 0.5 | 16.1 ± 2.9 |
| DMN + Oltipraz 5 mg/kg | 3.1 ± 0.4* | 11.1 ± 1.7* |
| DMN + Oltipraz 15 mg/kg | 2.9 ± 0.8* | 12.1 ± 1.9* |
| DMN + Oltipraz 50 mg/kg | 2.5 ± 0.9** | 8.0 ± 1.6** |

Each value is represented by the average±standard deviation. The number of animals used was 8 to 16. The significance of each group is determined by the Newman-Keuls test of multiple analysis. *p<0.05, **p<0.01. Degree of fibrosis 0=Normal, 1=Presence of weak fibrous tissue, 2=Moderate presence of fibrous tissue, 3=Obvious presence of fibrous tissue, 4=Evidence of severe fibrosis. Sum of values from periportal bridging (Greatest=10), intralobular cell loss (Greatest=4), portal inflammation (Greatest=4), and fibrosis (Greatest=4) yields the Knodell score.

### Test Example 3

### Pharmacological Mechanism of Oltipraz in Anti-Fibrosis

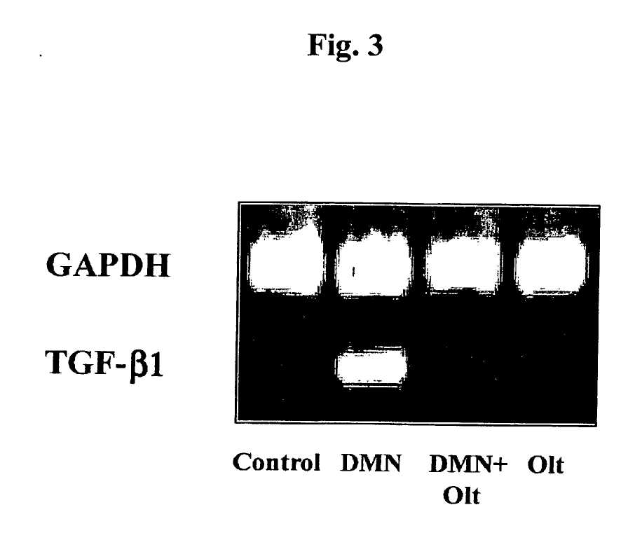

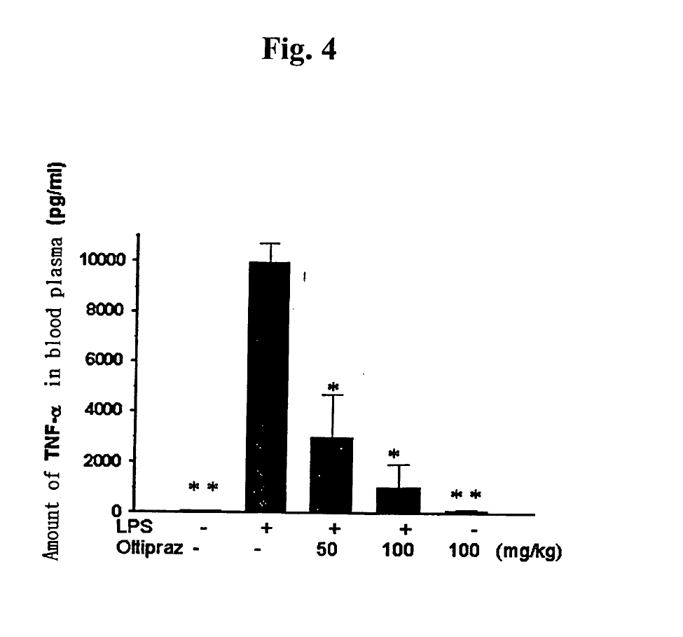

TGF-β 1 is a principal cytokine that rises in expression during fibrosis due to tissue damage. Animal TGF-β 1 mRNA expression was observed under RT-PCR analytical methods during DMN-only administration and DMN and oltipraz simultaneous administration. In animals administered with DMN over 4 weeks, the expression of TGF-β 1 mRNA was not observed due to irreversible excess fibrogenesis. The expression of TGF-β 1 mRNA was assessed after treatment of animals with a single dose of DMN. 18 hours after DMN administration, oltipraz was administered and TGF-β 1 mRNA expression was then observed 24 hours later. In DMN administered rats, TGF-β 1 mRNA increased notably in liver tissue. DMN induced expression of TGF-β 1 mRNA was completely inhibited by the administration of 100 mg/kg oltipraz. GAPDH mRNA expression did not change upon either DMN-only administration or simultaneous administration of DMN and oltipraz. Therefore, it is shown that oltipraz inhibits hepatic fibrosis through the pharmacological mechanism that reduces TGF-β 1 expression (FIG. 3).

### Test Example 4

### Evaluation of Oltipraz in Inhibiting TGF-β Production

A test was conducted using RAW264.7 macrophage cells to observe whether oltipraz directly inhibited production of TGF-β, which is over-expressed in macrophages, and to evaluate related molecular pharmacological mechanisms. When oltipraz was added to RAW264.7 cells expressing TGF-β, oltipraz inhibited the expression of TGF-β in a dose-dependent manner. These results show that oltipraz may function as an anti-fibrotic agent in hepatic Kupffer cells by inhibiting TGF-β production. Furthermore, the increase in TGF-β expression is inhibited by EGTA or genistein, which is an inhibitor of tyrosine kinase. This result shows that the inhibition of TGF-β production by oltipraz may be the result of intracellular calcium regulation and changes in protein kinase activity (Table 3).

TABLE 3
Inhibition of TGF-β Expression by Oltipraz in Macrophages

| | | Oltipraz | Oltipraz | EGTA | Genistein |
|---|---|---|---|---|---|
| | Control | 30 μM | 100 μM | 1 mM | 100 μM |
| TGF-β | 0 | 30 | 60 | 80 | 80 |
| Inhibition | | | | | |
| Percentage (%) | | | | | |

### Test Example 5

### Inhibition of TNF-Alpha Production by Oltipraz

TNF-alpha, a cytokine released from macrophages, plays a role in host defense mechanism by killing microbes like bacteria. However, when TNF-alpha is excessively produced, the amplified inflammatory response induces cell death. This is a general basis for utilizing anti-TNF-alpha antibodies or inhibitors of TNF-alpha production for treatment of systemic inflammatory diseases. In the present test, in order to confirm whether oltipraz inhibits the activity of Kupffer cells, the effect of oltipraz on TNF-alpha production was observed in endotoxin (LPS)-administered rats. When oltipraz was administered to the rats, TNF-alpha production increased by LPS was inhibited in a dose-dependent manner. The phenomenon in which oltipraz inhibits TNF-alpha production suggests that oltipraz also inhibits the inflammatory response of hepatic tissue and that the cells, on which oltipraz acts, are Kupffer cells. Along with the inhibition of TGF-β production, the inhibition of liver inflammatory response may be a mechanism by which oltipraz shows protective effects on hepatic tissue (FIG. 4, *, **; Significance compared to LPS administered animal group; p<0.05, p<0.01).

### WORKING EXAMPLES

### Working Example 1

| | |
|---|---|
| Oltipraz | 25 mg |
| Lactose | 50 mg |
| Starch | 10 mg |
| Magnesium stearate | Proper amount |

The above components are mixed and a tablet is prepared by means of a conventional tablet preparation process.

### Working Example 2

| | |
|---|---|
| Oltipraz | 100 mg |
| Lactose | 50 mg |
| Starch | 10 mg |
| Magnesium stearate | Proper amount |

The above components are mixed and a tablet is prepared by a conventional tablet preparation process.

### Working Example 3

| | |
|---|---|
| Oltipraz | 250 mg |
| Lactose | 50 mg |
| Starch | 10 mg |
| Magnesium stearate | Proper amount |

The above components are mixed and a tablet is prepared by means of a conventional tablet preparation process.

### Working Example 4

| | |
|---|---|
| Oltipraz | 25 mg |
| Lactose | 30 mg |
| Starch | 28 mg |
| Talc | 2 mg |
| Magnesium stearate | Proper amount |

The above components are mixed and a capsule preparation is prepared by filling a hard gelatin capsule with this mixture through a conventional capsule preparation process.

### Working Example 5

| | |
|---|---|
| Oltipraz | 100 mg |
| Lactose | 30 mg |
| Starch | 28 mg |
| Talc | 2 mg |
| Magnesium stearate | Proper amount |

The above components are mixed and a capsule preparation is prepared by filling a hard gelatin capsule with the mixture through a conventional capsule preparation process.

### Working Example 6

| | |
|---|---|
| Oltipraz | 250 mg |
| Isomerized sugar | 10 g |
| Sugar | 30 mg |
| Sodium CMC | 100 mg |
| Lemon Flavor | Proper amount |

(add purified water to total volume of 100 ml)

A suspension is prepared with the above components according to conventional suspension production methods. A 100 ml brown bottle is filled with the suspension and sterilized.

### Working Example 7

| | |
|---|---|
| Oltipraz | 500 mg |
| Isomerized sugar | 20 g |
| Sugar | 20 g |
| Sodium arginate | 100 mg |
| Orange flavoring | Proper amount |

Add purified water to 100 ml

A suspension is prepared with the above components according to conventional suspension production methods. A 100 ml brown bottle is filled with the suspension and sterilized.

### Working Example 8

| | |
|---|---|
| Oltipraz | 250 mg |
| Lactose | 30 mg |
| Starch | 20 mg |
| Magnesium stearate | Proper amount |

The above components are mixed and filled in a polyethylene coated envelope and sealed to prepare a powder.

### Working Example 9

1 Soft capsule containing

| | |
|---|---|
| Oltipraz | 100 mg |
| Polyethylene glycol | 400 mg |
| Concentrated glycerin | 55 mg |
| Purified water | 35 mg |

Polyethylene glycol is mixed with concentrated glycerin, and then purified water is added. Maintaining the mixture at 60° C., oltipraz is added to the mixture. The mixture is stirred at approximately 1,500 rpm. After the mixture has been combined uniformly, the mixture is cooled at room temperature while being slowly stirred. Air bubbles are removed with a vacuum pump, leaving the contents of the soft capsule.

The soft capsule membrane is manufactured according to conventional preparation methods using a widely known soft gelatin-plasticizer formula containing gelatin 132 mg, concentrated glycerin 52 mg, 70% disorbitol solution 6 mg per capsule, a proper amount of ethyl vanillin flavoring agent, and carnauba wax as the coating agent.

The pharmaceutical composition comprising oltipraz according to the present invention exhibit surprisingly excellent effect on the treatment and prevention of liver fibrosis and cirrhosis.

What is claimed is:

1. A use of 5-(2-pyrazinyl)-4-methyl-1,2-dithiol-3-thione (oltipraz) as a medicine for preventing and treating the progression of hepatic fibrosis and cirrhosis.

2. A pharmaceutical composition for preventing and treating the progression of hepatic fibrosis and cirrhosis, comprising 5-(2-pyrazinyl)-4-methyl-1,2-dithiol-3-thione and a pharmaceutically acceptable excipient.

3. A pharmaceutical composition according to claim 2, wherein the composition is formulated as a form selected from a group consisting of a capsule, a tablet, a soft capsule, a suspension, a syrup, an injection, and a powder.

4. A pharmaceutical composition according to claim 2, wherein the composition is for oral administration.

5. A use of 5-(2-pyrazinyl)-4-methyl-1,2-dithiol-3-thione for the manufacture of a medicine for preventing and treating the progression of hepatic fibrosis and cirrhosis.

6. A method for preventing and treating the progression of hepatic fibrosis and cirrhosis comprising administering the pharmaceutical composition of claim 2 to mammals.
